(19) United States
(12) Patent Application Publication
(10) Pub. No.: US 2025/0366738 A1
(43) Pub. Date: Dec. 4, 2025

(54) HEARING CORRECTION SYSTEM

(71) Applicant: MELISONO AB, Lund (SE)

(72) Inventor: Bernt BÖHMER, HÖÖR (SE)

(21) Appl. No.: 18/704,692

(22) PCT Filed: Nov. 9, 2022
(86) PCT No.: PCT/SE2022/051044

§ 371 Date: Apr. 25, 2024

(30) Foreign Application Priority Data
Nov. 10, 2021 (SE) 2151378-3

(51) Int. Cl. A61B 5/12; H04R 25/00

(52) U.S. Cl. CPC A61B 5/121; H04R 25/453; H04R 25/505; H04R 25/70

(57) ABSTRACT

The presented novel Hearing Correction System comprises a device and method for a hearing test to assess hearing loss, software to process the measured data and provide settings to digital signal processing elements and a digital signal processing topology to perform dynamic compensation. The new hearing test is based on the impression of equal loudness and measures hearing ability at several sound pressure levels, not just at the hearing threshold as done by the standard audiometry test.

### INTRODUCTION

By the end of World War II, Raymond Carhart and Norton Canfield started aural hospitals for soldiers who were experiencing hearing loss. At these hospitals, they developed the electronic audiometer used for hearing tests. The results from the audiometer tests were referred to as audiograms. Whilst technology and technical equipment has been vastly improved since the end of World War II the basic function of the audiometer remains the same until present day.

The presently most common hearing test for assessing hearing loss and fitting of hearing aids is called pure tone audiometry, which measures the threshold of hearing for both ears individually at eight standard frequencies from 250 Hz to 8000 Hz. The result of the test is displayed in an audiogram. The audiogram shows the threshold of hearing at each of the tested frequencies.

Interviewing hearing aid users and people who experience hearing loss that have tried using hearing aids, a pattern emerges. Many express dissatisfactions with the amplification of sound from the hearing aid. Most regular hearing aid users say that they are helped with dialog intelligibility in environments with low to moderate sound pressure but generally think that at slightly higher sound pressure levels the sound from the hearing aid becomes too loud particularly at higher frequencies, brittle, and metallic. People who have tried hearing aids but opted to not use them think that the loud, brittle, and metallic sound is unbearable, and improvements if any are outweighed by this considerable drawback. It seems like a rather severe hearing loss is normally required for the user to accept the ubiquitous drawbacks of loud brittle sound at higher frequencies.

The relevance of the common audiometer test and the audiogram relies on the assumption that the human hearing mechanism is essentially exhibiting a linear response to input. An elevated hearing threshold by say 20 dB at a certain frequency is assumed to give a proportionate attenuation of 20 dB throughout the entire audible dynamic range at that frequency. This is evidently not entirely true since hearing aids are usually not programmed to fully compensate for the loss of hearing exhibited by the hearing threshold measurements in audiograms.

There are a significant number of scientific papers and published philosophies covering the subject of compensation level in relation to audiogram data. A very rudimentary summation provides the conclusion that around half to two thirds of the measured degradation should be compensated depending on circumstances.

Considering the apparently weak connection between measured elevated hearing threshold and optimal applied compensation together with the less-than-ideal user experience feedback, it seems there is reason to suspect that the presently used measurements and compensation for hearing loss in hearing aids is less than optimal and a new methodology is required.

The presented novel Hearing Correction System comprises a hearing test to assess hearing loss, software to process the measured data and provide settings to digital signal processing elements and a digital signal processing topology to perform dynamic compensation. The hearing test measures hearing ability at several sound pressure levels, not just at the hearing threshold, and is based on the impression of equal loudness. The digital signal processing topology utilizes dynamic elements that provide dynamically varying amplification based on measured hearing loss and momentary sound pressure level.

### SUMMARY OF THE PRESENT INVENTION

The present invention is directed to a device for hearing assessment and/or correction, said device comprising a software unit arranged for performing a method comprising
performing a hearing test with different frequencies and with different sound levels on a test subject, said hearing test involving using a reference sound and a test sound and adjusting the test sound level until the test subject perceives the test sound level to be more or less equal to the reference sound in loudness; and
obtaining data on the test sound level and the reference sound for the different frequencies at different sound levels,
and wherein one or more dynamic filters are involved for compensating the data.

In relation to the above it should be noted that the device according to the present invention may be of any type of device suitable to include a software unit, such as a mobile phone, PC or other computer unit, tablet etc. Moreover, personal sound units, such as personal sound amplifier products are also such embodied by the present invention.

### DETAILED DESCRIPTION OF THE HEARING CORRECTION SYSTEM

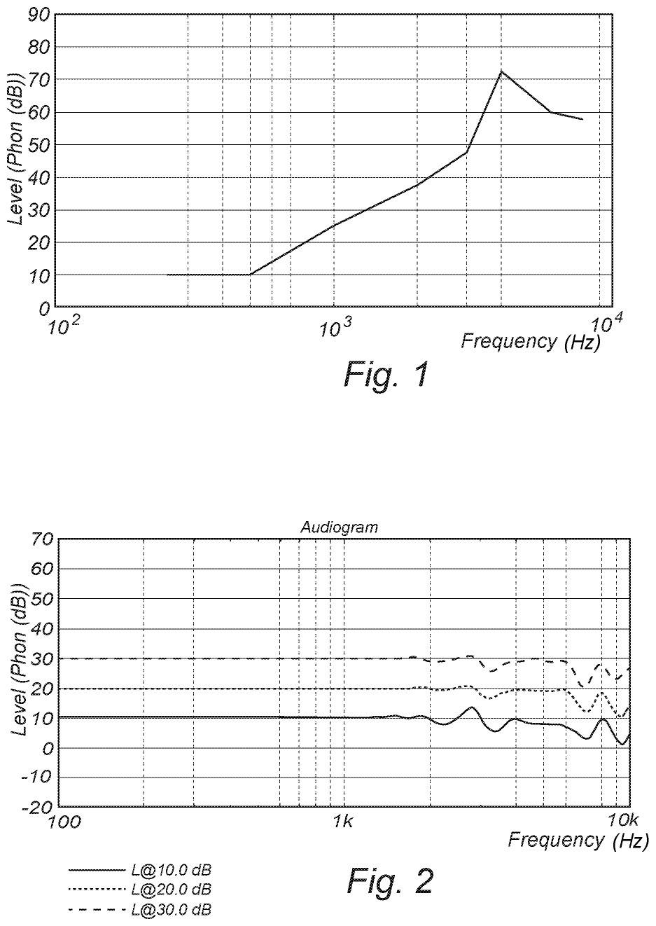

The common pure tone audiometry test produces an audiogram with hearing threshold data for each ear. The relevance of the test for assessing hearing loss is based on the assumption that an elevated hearing threshold translates to a perceived reduction of level corresponding to the measured elevation. In other words, if the hearing threshold is elevated by 20 dB at a particular frequency there is an assumption that there is a corresponding perceived attenuation of sound by 20 dB across the entire audible dynamic range at that frequency. FIG. 1 shows hearing threshold data from an audiogram for a person with significant hearing loss at the 8 standard frequencies (250 Hz, 500 Hz, 1 kHz, 2 kHz, 3 kHz, 4 kHz, 6 kHz and 8 kHz) starting at 250 Hz shown to the left in the diagram up to 8 kHz to the right.

The new Hearing Correction System uses a novel hearing test that assesses hearing loss across a broad dynamic range which is significantly different from the pure tone audiometry test that only provides information about the hearing threshold. The test is based on perceived Equal Loudness of a reference sound compared with a test sound. The test sound level is adjusted until the test person perceives it to be equal to the reference sound in loudness. Typically, the reference sound and test sound are alternatingly played back at an alternation frequency of 0.5 Hz. The alternation frequency can obviously be changed both to a higher or a lower frequency. A feasible range would be from 0.1 Hz to 2.5 Hz. Alternation between the two compared tones could be automatic as well as under manual control.

The equal loudness levels used are based on the ISO226 standard's levels at different frequencies and sound pressure. Test and reference sound can be pure sine tones like the audiometry test, or it could be other types of sounds, bandwidth limited to the desired test frequency band. Such sounds could as an example be made up of warble tones, noise, or any type of complex signals containing a multitude of frequencies. Any type of signal that has an appropriate bandwidth to fit the test frequency band could be used.

In the standard audiometry test the frequency range is divided into the eight well known standard test frequency bands. The Equal Loudness test results shown in the figures uses 81 frequencies in the range between 100 Hz and 10 kHz. This many test frequencies are of course not crucial, the number of frequencies tested can be anything between 1 up to the presented 81 or even more. Preferably the number of test frequencies should be in the range 5 to 35. Fewer test frequencies produce a faster test that is easier for the test subject to perform but will also provide less information and a suitable balance is necessary. Each individual test frequency can be either a pure sine tone or a complex signal with most of its frequency components contained within the test frequency band in between adjacent test frequencies. Complex test signals with a wider frequency spectrum than a pure sine can provide more information about hearing ability within the test frequency band and are particularly useful in tests with fewer test frequencies. The Equal Loudness test frequency range can be extended to all frequencies covered in the IS226 standard i.e., below 100 Hz down to 20 Hz and above 10 KHz up to 12.5 kHz. The test frequency range can of course be further extended if equal loudness data is available and there is a desire to test an even broader frequency range than 20 Hz to 12.5 kHz. Under normal conditions the range 100 Hz to 12.5 kHz is preferred and in many cases, it can be reduced to the range between 100 Hz and 10 KHz. Further reduction to as an example the audiometry test frequency range 250 Hz to 8 kHz is also purposeful.

The Equal Loudness test is typically performed at sound pressure level intervals of 10 dB where the lowest test level should be just above the test subject's hearing threshold at the reference frequency. Any other sound pressure level granularity can be used both more closely spaced for example 5 dB or 3 dB and wider like 15 dB or 20 dB. Usually, the lowest test level is set at 10 Phon if the test subject has no to low hearing loss at the reference frequency. If the test subject experience hearing loss at the reference frequency the lowest level can be increased. The Equal Loudness test is usually limited to a maximum sound pressure level of 70 Phon to avoid subjecting the person undergoing the test to sound pressures that could cause hearing damage. There is of course no other limitation restricting the highest sound pressure during the test but the 70 Phon level seems to be the highest level that most test subjects feel comfortable being subjected to and is within safe limits.

The Equal Loudness test should be carried out at a test level that is just above the test subject's hearing threshold i.e., 0-10 dB above preferably less than 5 dB, at the reference frequency and minimum one more higher sound pressure level. More sound pressure test levels offer a better assessment of the test subject's hearing ability. At least two sound pressure test levels are required and normally four levels with a sound pressure level spacing of 10 dB produce good results. Testing at more levels increase the burden put on the test subject to carry out the test and it is therefore desirable to test at fewer sound pressure levels. Three test levels with a sound pressure level spacing of 10 dB constitutes a reasonable compromise in many cases producing adequate results. The number of levels required is dependent on the sound pressure level spacing between the tests. Lower spacing requires more tests to produce good data and larger spacing fewer. The number of tests could of course be any number from two and up.

At first, it is necessary to make sure that the test subject can hear the reference tone at the lowest level used in the test. An ordinary hearing test establishing the hearing threshold at the selected reference frequency is used for this purpose. If it is found that the test subject suffers from severe hearing loss at the reference frequency it may be beneficial to select another reference frequency where the hearing loss is less severe. A reference frequency should be located approximately in the middle of the frequency range to be examined since it makes the Equal Loudness comparison easier for the test subject. However, if hearing loss in the middle of the frequency range is severe moving the reference to another frequency might provide a useful compromise. Another technique to make comparisons easier is a stepped approach utilizing multiple reference frequencies. Multiple reference frequencies reduce the distance in frequency between the test and reference tones which makes comparisons easier. Whereas the basic one reference tone test aim to put the reference tone in the middle of the frequency range a stepped approach uses at least two reference tones, but can be any number, with the reference tones about evenly spread across the frequency range under test. With a stepped approach the Equal Loudness levels of the reference tones are first tested so that all reference tones are perceived to have the same loudness starting with a reference tone located at a frequency where the hearing loss is as small as possible. Then the test tones are compared with one or more of the reference tones to complete the test.

FIG. 2 displays Equal Loudness test data for a normal hearing person at the levels 10 Phon, 20 Phon and 30 Phon. The test was carried out with a reference tone at 880 Hz, and test tones varied from 100 Hz up to 10 kHz in 81 steps. The reference and test tones alternated automatically with 0.5 Hz. There are some wiggles present in the graphs at all levels at frequencies above approximately 1.8 kHz. These smaller wiggles do not signify a hearing loss or hearing deviation. They are a combination of normal variations in headphone response at higher frequencies experienced by the individual and the test subject's specific deviations from the ISO226 average loudness data. Headphone response variations are normal and occur due to the headphone's interaction with the outer ear and ear canal of the test subject. The only very slight deviation indicating minimal hearing threshold increase is the small peak in the 10 Phon graph just below 3 kHz. The perceived level difference between 10 Phon, 20 Phon and 30 Phon are all 10 dB which is indicated by the even spacing of 10 dB between the graphs at all frequencies. Evidently, the assumption and basis for the audiometer test, that the human hearing mechanism is essentially exhibiting a linear response to input level changes is true for a normal hearing person. This is however not the case for people suffering from hearing loss, which will be shown later.

FIG. 3 shows audiogram data for the eight standard frequencies for a person with significant hearing loss (blue graph (2)) compared with Equal Loudness test data for the same person (red graph (1)) at the lowest Equal Loudness test level which is slightly above the hearing threshold. Apart from local small variations the Equal Loudness data and audiogram data correlate well verifying the efficacy of the Equal Loudness test.

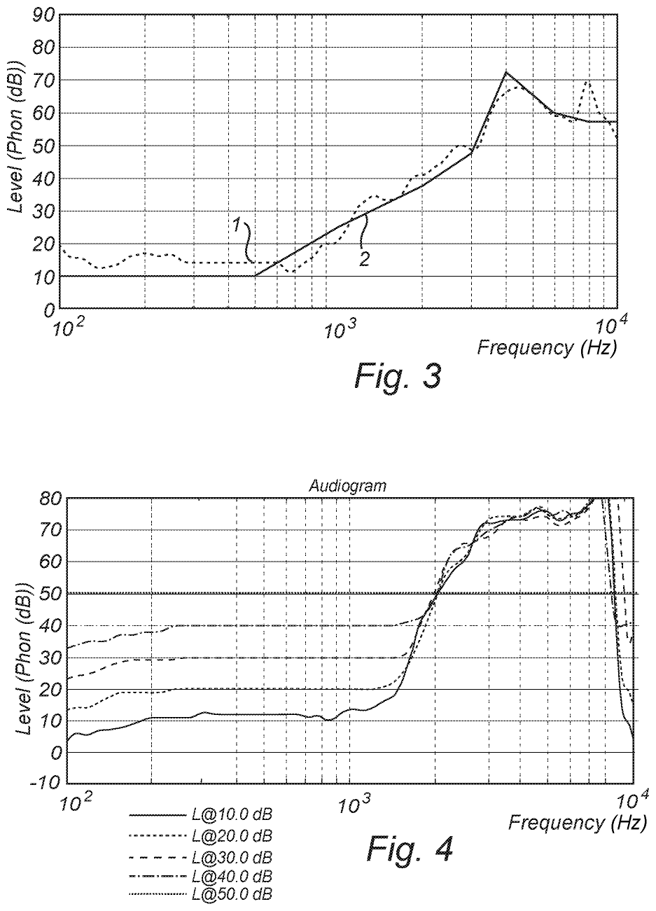

FIG. 4 reveals Equal Loudness test data at levels 10 Phon, 20 Phon, 30 Phon and 40 Phon for a person with significant high frequency hearing loss. The bottom trace shows the lowest 10 Phon level which is just above or at the test subject's hearing threshold at the reference frequency of 880 Hz. The graphs display to the right a significant elevation of the hearing threshold starting just above 1 kHz. To the left in the diagram, at lower frequencies, the graphs are spaced by the expected 10 dB. At higher frequencies beginning from approximately 2 kHz and above, the spacing is significantly less than 10 dB. At 300 Hz the 10-40 Phon traces are separated by almost 40 dB, which corresponds to the sound pressure level increase. The slight elevation of the 10 Phon trace between 100 Hz-1 KHz produces a slightly smaller separation to the 20 Phon trace than between the other traces. This is caused by a slight elevation of the hearing threshold influencing at the lowest 10 Phon level. The elevation disappears at 20 Phon and above where the spacing is exactly 10 dB apart from a few small local deviations. At higher frequencies above 2 kHz where there is significant elevation of the hearing threshold the 10-40 Phon traces are separated by just a few decibels. This reveals that at say 3 kHz the sound pressure level just needs to increase by a couple of decibels to be perceived by the test subject to have increased by 40 dB. Very clearly the brain starts to radically compensate for the elevated hearing threshold once the sound pressure level is above the hearing threshold and the ear/brain is no longer behaving as a linear device, actually very far from it in this case.

This will obviously create a massive problem if the elevated hearing threshold were to be compensated with a hearing aid fitted using the present standard methods relying on the assumption of linear hearing behavior assessed only by looking at the hearing threshold. With a standard compensation from approximately half to two thirds of the measured hearing threshold elevation the sound would at lower levels and frequencies above approximately 2 kHz not be amplified enough and at higher levels amplified far too much.

The test subject in FIG. 4 has got professionally fitted hearing aids but is not using them since they are perceived to not help with speech intelligibility at lower levels and at somewhat higher levels amplify higher frequencies far too much. The hearing test data shown in FIG. 4 clearly support the subjective analysis from the test subject and provide an objective explanation to the subjective assessment and reported problems.

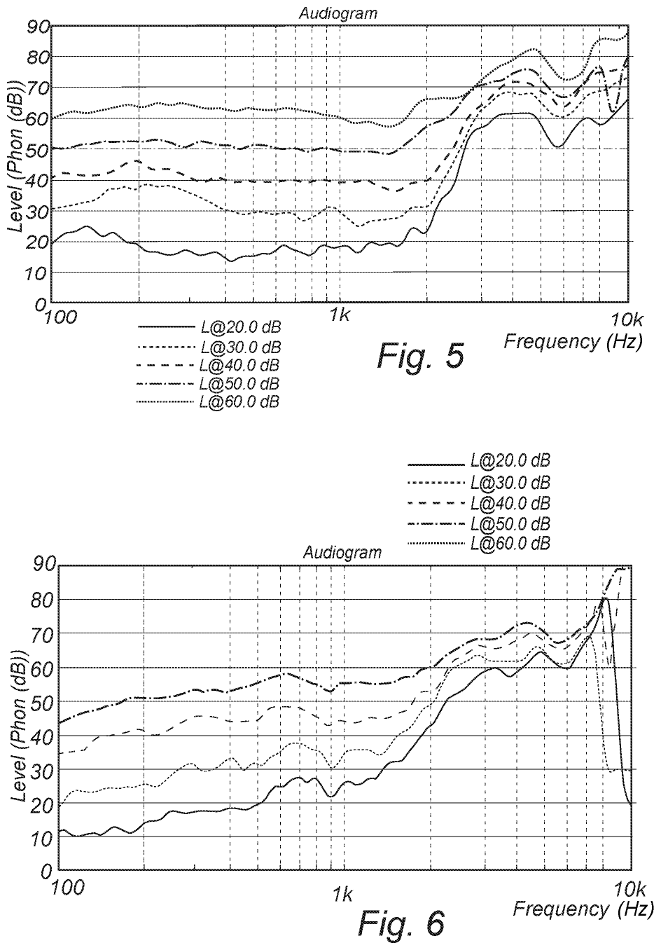

Normally the dynamic range compression at frequencies with an elevated hearing threshold is smaller than the case presented in FIG. 4, but all test subjects show similar dynamic compression at frequencies where there is an elevation of the hearing threshold. Looking at all our collected measurement data it is obvious that the brain automatically compensates for an elevated hearing threshold at higher sound pressure levels when the sound is above the hearing threshold and consequently can be heard. The brain's compensation can be quite dramatic as in FIG. 4 or smaller, but it is always present to a significant degree. FIGS. 5, 6 and 7 show Equal Loudness test data from three more test subjects with similar compression behavior. The measured compression at higher frequencies where there is hearing loss is still very significant but not quite as dramatic as in FIG. 4.

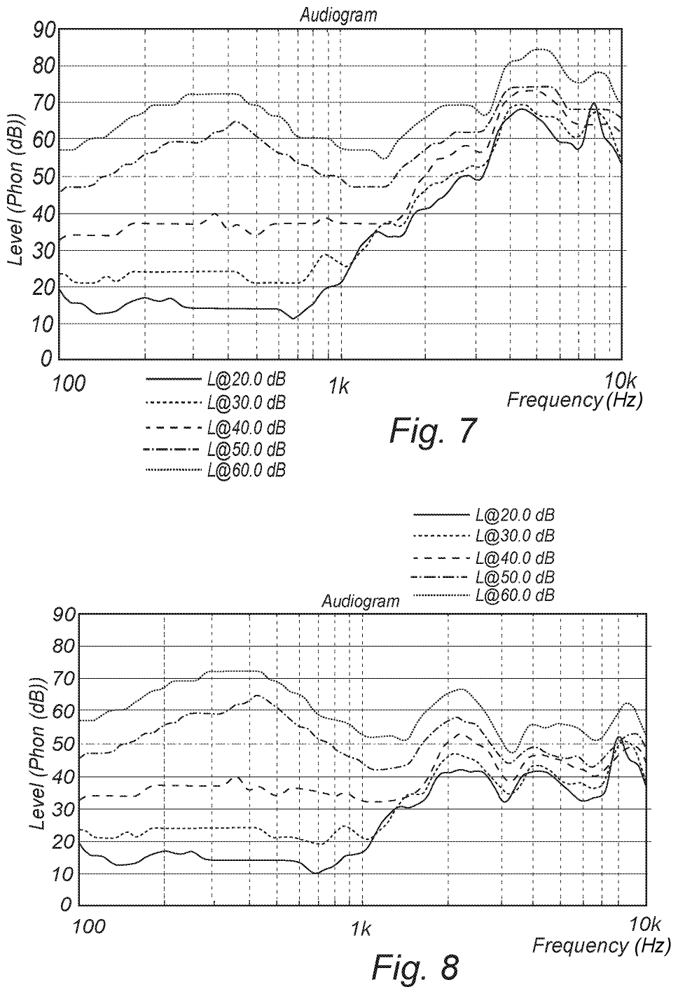

The Equal Loudness test data displayed in FIG. 7 originates from a test subject that has got hearing aids professionally fitted and wears them daily. As can be seen in the figure the person has a significant hearing loss at higher frequencies and the usual compression of the dynamic range is present. The Equal Loudness test data also reveals a somewhat unusual hearing loss at higher levels, 50 and 60 Phon, in the lower frequency range approximately between 150 Hz to 800 Hz. The standard audiometer test and audiogram would not reveal this loss, nor would it provide any information about the degree of dynamic compression at higher frequencies. The audiogram data shown in FIGS. 1 and 3 originates from the same test subject as does the lowest level Equal Loudness test data shown in FIG. 3. The full Equal Loudness test data is shown in FIG. 7.

The Equal Loudness test data shown in FIG. 8 is from the same test subject as in FIG. 7, but the Equal Loudness test was carried out whilst the test subject was wearing a professionally fitted hearing aid. A comparison between the traces in FIGS. 7 and 8 make it clear that the hearing aid increasingly amplify higher frequencies more and more starting at approximately 1 kHz. The 20 Phon trace in FIG. 8 at 4-5 kHz when compared with the same trace in FIG. 7 suggest an amplification of about 25 dB at these frequencies.

The hearing aid's amplification is too low at 20 Phon seen by the gradual rising level for frequencies above 1 kHz and the compensation is in this case about half of the measured hearing threshold increase displayed in the audiogram in FIG. 3. Amplification at the highest sound pressure level in the Equal Loudness test, 60 Phon, is too high indicated by the gradual downward slope of the trace at higher frequencies at that level. At higher sound pressure levels than 60 Phon, which is a moderate sound pressure level experienced in real life, the downward slope would increase even further, and the high frequency amplification would be far too high.

If the hearing aid restored the hearing of the test subject to normal the Equal Loudness test data would look like the data from a normal hearing person shown in FIG. 2. Clearly the Equal Loudness test data in FIGS. 2 and 8 are very dissimilar and the hearing aid has failed to properly compensate for the hearing loss experience by the test subject. The test subject delivers the same feedback on the hearing aid experience as many other hearing aid users i.e., too little amplification of higher frequencies at lower levels and excessive high frequency amplification at higher sound pressure levels. The person still uses the hearing aids daily since the hearing loss is significant and they help restoring speech intelligibility but provide too high amplification at higher frequencies of louder sounds. The Equal Loudness test data objectively explains the subjective experience.

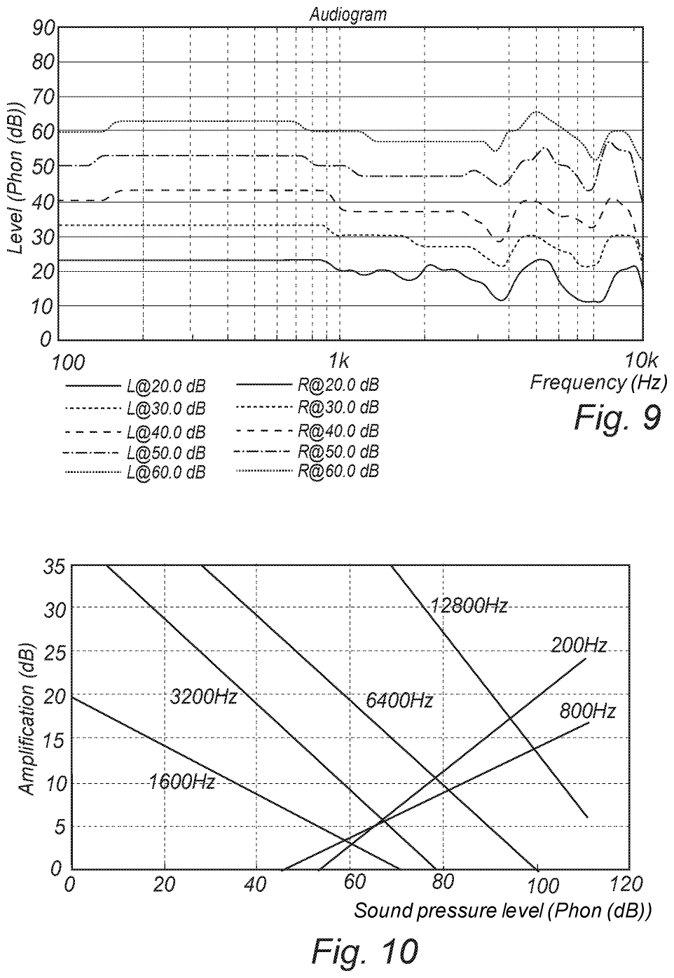

FIG. 9 is again Equal Loudness test data from the same test subject as FIGS. 7 and 8 but now the hearing loss has been dynamically compensated by the DSP part in the novel Hearing Correction System using input data derived from the Equal Loudness test shown in FIG. 7. Comparing FIG. 9 to the Equal Loudness test data from the normal hearing person shown in FIG. 2 it is apparent that the correction is very good and the test subject's hearing ability has essentially been restored to normal hearing. This is unmistakably a vast improvement on the results from the regular hearing aid displayed in FIG. 8.

FIG. 9 shows that the unusual hearing loss at higher levels, 50 and 60 Phon, in the lower frequency range approximately between 150 Hz to 800 Hz, has been removed almost entirely by the novel Hearing Correction System with only a small trace left. The high frequency compensation above 1 kHz is now linear the lowest 20 Phon level is properly amplified above 1 kHz and at the same time amplification at higher levels is reduced resulting in flat responses equal to results for a normal hearing person at all levels. The traces are also evenly spaced by approximately 10 dB with the dynamic compression removed.

Test subjects' subjective reactions to the experience of using the Novel Hearing Correction system is exceptionally positive and it is reported to be very natural sounding without negative influences from the amplification. Speech intelligibility is greatly enhanced without shortcomings and the experience of sound is likened to wearing glasses, everything is significantly clearer with more audible detail and all in all much better without perceptible downsides.

The compensation used to obtain Equal Loudness test data shown in FIG. 9 uses 6 dynamic filters between 200 Hz and 12.8 kHz. The filters' center frequencies are 200 Hz, 800 Hz, 1.6 kHz, 3.2 kHz, 6.4 kHz and 12.8 kHz. The number of filters required to compensate a hearing loss varies from individual to individual and any number of filters from only one up to several hundred is conceivable but in practical applications the range 1 to 20 filters is preferred. Most normal corrections can be solved using between 1 to 8 filters. The center frequencies of the filters can of course also be varied to suit the required correction. The stated center frequencies are all multiples of two missing 400 Hz that wasn't required in this particular case. An even spread of center frequencies is not necessary but preferable since the filters will normally overlap to some degree and interaction between the filters' amplification need to be considered when calculating the composite amplification from all filters. An even spread makes the calculations easier. The multiplication factor of 2 between center frequencies is just an example of a useful and suitable number which can be changed to any other value.

Software algorithms in the Hearing Correction System use the Equal Loudness test data shown in FIG. 7 to produce input to the DSP part performing the compensation to achieve the Equal Loudness test data shown in FIG. 9. Maximum amplification level at each filter's center frequency is determined and aggregate amplification from adjacent filter bands are considered. Maximum amplification is also limited by the algorithms to avoid acoustic feedback in case the target device is a hearing aid where acoustic feedback can occur. In pure playback applications where there is no microphone feedback path and consequently no risk of feedback this is not necessary. Tables produced by the software algorithms for each filter frequency are used by the DSP part to determine the dynamically changing amplification. FIG. 10 shows filter table data output from the algorithms for each filter used to achieve the compensation in FIG. 9.

Input sound pressure level is displayed in Phon on the X-axis and amplification in dB on the Y-axis in FIG. 10. Looking at the graph showing 1600 Hz table data it can be seen that the maximum amplification at 0 Phon is slightly less than 20 dB. The amplification is gradually reduced and arrive at 0 dB at approximately 70 Phon i.e., no amplification. Input sound pressure level is measured in each filter band individually i.e., if there is a pure tone at 1600 Hz predominantly the amplification at 1600 Hz will be influenced. At 0 Phon the 3200 Hz table data is at a maximum of 35 dB which in this case is set to avoid acoustic feedback. The 35 dB limit can of course be both higher and lower depending on the used hearing aid's feedback properties. The limit can also vary from frequency to frequency but in the example, it is set to 35 dB for all frequencies. The 35 dB value is a reasonable value that can be achieved by an average modern hearing aid with fair feedback cancellation. The 3200 Hz amplification table data is 0 dB at a slightly higher level than the 1600 Hz table, just below 80 Phon. The 6400 Hz and 12800 Hz table data is like 1600 Hz and 3200 Hz albeit exhibiting higher amplification relative to input sound pressure level. Table data for 200 Hz and 800 Hz is different in that it instead of decreasing amplitude gradually with increasing input sound pressure level is increasing amplification with increasing input sound pressure level. This is necessary to compensate for the hearing loss at the lower frequencies at higher sound pressure levels shown in FIG. 7.

A typical hearing aid implementation also requires a limiter applied in the DSP on the output to reduce maximum sound pressure. The limiter will prevent risk of hearing damage keeping reproduced sound pressure within safe limits and limit levels to stay within the output device's dynamic reproduction range.

Table data for each frequency is calculated from the Equal Loudness test data shown in FIG. 7. The input sound pressure is amplified so that the experienced level is fully compensated if applicable within the limits set by acoustic feedback. Essentially table data is collected by drawing a straight vertical line at the particular center frequency in FIG. 7 creating table data from deviations between experienced level and real level as noted along the vertical line for each input sound pressure level. Since the measured Equal Loudness test data's upper level is limited, table data for sound pressures above the test's limit must be extrapolated. One possible way of extrapolating data is through polynomial fitting to available test data and a first order polynomial fit has been used in practical tests with good results. Obviously, there are numerous ways to extrapolate the extended data and a polynomial fit is just an example that have proved effective.

A more exact method comprising multiple steps is however preferred. Acquired hearing data is somewhat irregular, varying up and down, which as an example can be seen by the wiggles on the traces in FIGS. 4 to 6. The wiggles are partly due to frequency deviations in the measurement setup but also related to human errors and uncertainty in the test. The variation of the dynamic compression seen to the right at the higher frequencies in these figures exhibit irregular compression varying up and down from the trend depending on frequency and level. If many iterations of tests are made these variations even out and disappear but practically this is not possible. The test procedure is mentally arduous for the test person and after a while mental fatigue sets in and accuracy is consequently degraded. To regain accuracy the test person needs a significant break, and many iterations of tests would run the risk of extending the test period over several days, which obviously is not desirable and significantly suboptimal.

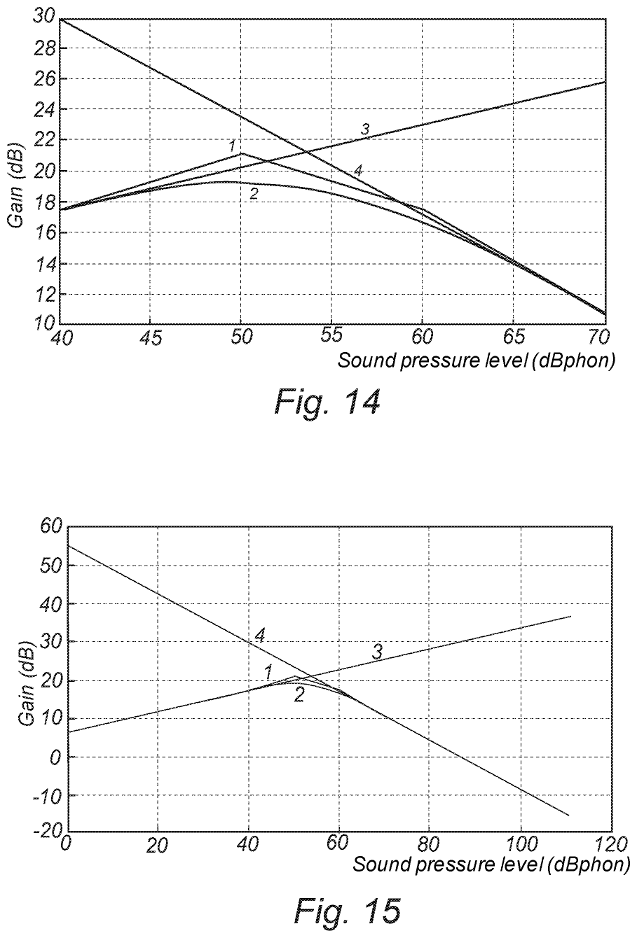

One method that is effective in a hearing restoration product is to maximize accuracy and make the most out of available data is described below. The method uses five steps. The first three steps can be understood by looking at the traces in FIG. 14. Trace 1 is connecting the four raw measurement data points, one at each 10 dB increment starting at 40 dB and ending at 70 dB. The first step is to extend the number of sample points far beyond the measured four points and even out variations in the measured data from a general underlying trend. There are many possible ways to do this mathematically and two possible solutions are to use cubic spline data interpolation or linear first order polynomial interpolation between the raw data points followed by a multiple order linear phase FIR average filtration of the new interpolated data. The interpolation can generate as many datapoints over the 40 dB-70 dB dynamic range as desired, in this case a 0.5 dB interval between data points is used. Trace 2 in FIG. 14 shows the extended and filtered data using linear interpolation and linear phase FIR filtering.

The second step is to extend the dynamic range below the lowest measured level, 40 dB. For this purpose, the first part of the interpolated and filtered data displayed by trace 2 is used. A straight-line derivative fit is made to the derivative at the beginning of the interpolated and filtered data. The straight line can then be used to extend the data below 40 dB. Trace 3 shows the straight line. Similarly in the third step, the last part of the interpolated and filtered data displayed by trace 2 is used to perform straight-line derivative fit to the derivative at the end of the interpolated and filtered data. The resulting straight line that can be used to extend data points above 70 dB is shown by trace 4.

FIG. 15 shows the whole dynamic range from 0 dB to 110 dB. Similar to FIG. 14, trace 1 is the measurement data points, trace 2 the extended and filtered data using linear interpolation and linear phase FIR filtering, trace 3 the fitted lower-level range straight line and trace 4 the fitted upper-level range. Looking at FIG. 15 it can be understood how trace 3 is used to extend the level range below 40 dB down to 0 dB and how trace 4 is used to extend the range above 70 dB. It can also be understood that a simple first order polynomial fit i.e., straight line, to the measured data would not have produced accurate results in this case. The measured data is collected from a person suffering from what is popularly called cookie bite hearing loss measured at 400 Hz. It is somewhat unusual in that less amplification is also required at lower levels not just higher as normally is the case. The data is verified to be accurate over several tests so the measurement although surprising is correct.

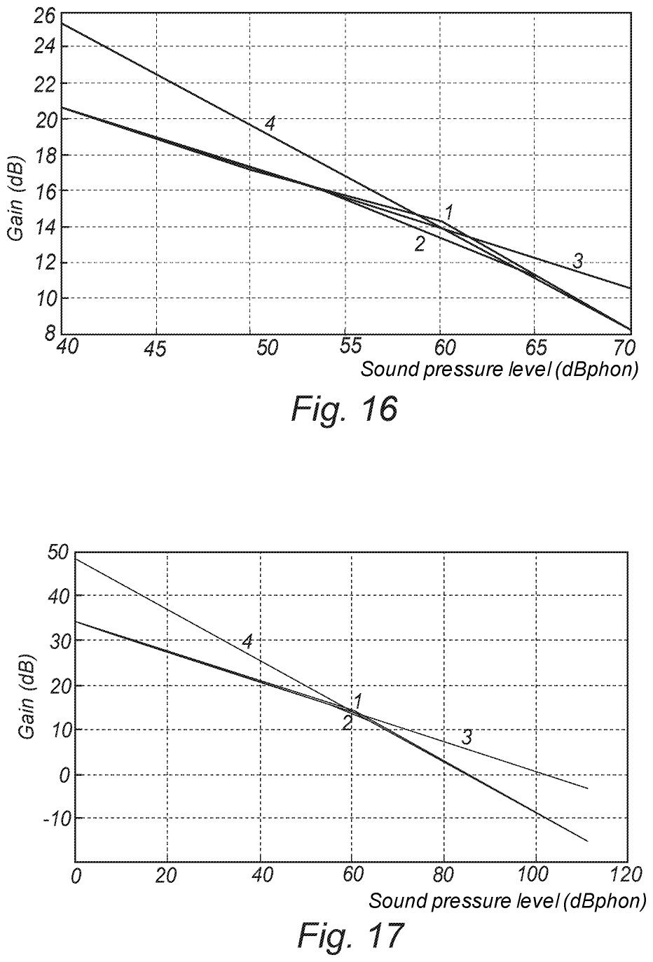

FIG. 16 shows another example of results from the method's first three steps. The data is measured at 2400 Hz between 40 dB and 70 dB at four levels. Again trace 1 is the measurement data points, trace 2 the extended and filtered data using linear interpolation and linear phase FIR filtering, trace 3 the fitted lower-level range straight line and trace 4 the fitted upper-level range. FIG. 17 shows the extended dynamic range from 0 dB to 110 dB for this data.

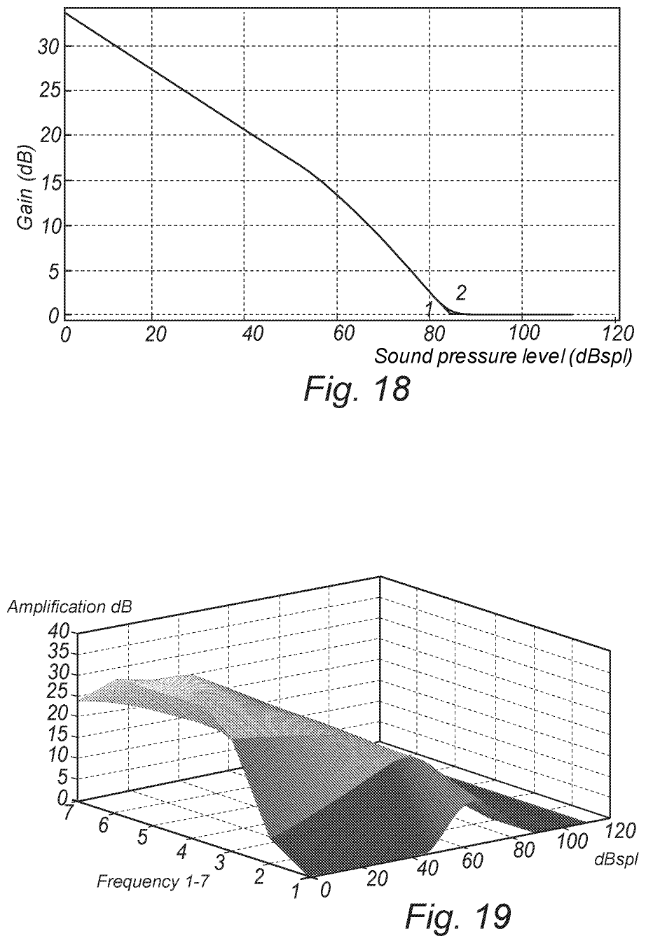

FIG. 18 displays the fourth step of the method. Gain required to restore the hearing experience is measured at Phon levels which relates to the human experience of loudness and not to the technical sound pressure level measured in dBspl. The sound pressure level is referenced to 20 μPa at all frequencies i.e., a sound pressure of 20 μPa is equal to 0 dBspl at all frequencies. 1 dBphon is defined to be equivalent to 1 dBspl at 1 KHz but the sound pressure level equivalent then varies greatly depending on frequency and sound pressure level. As an example 20 dBphon at 100 Hz equals approximately 28 dBspl. To create gain table data that is useful in the DSP, phon related data first must be mathematically transposed into spl related data. Trace 1 shows transposed gain table data across the whole dynamic range from 0 dB to 110 dB based on measured and extracted information with extracted data added below 40 dB and above 70 dB as described above. The table data set is limited to a minimum gain of 0 dB and maximum allowed gain. The maximum gain is normally set at a suitable level to avoid acoustic feedback but in this case, there was no need to apply an upper limit since the maximum required amplification only reached approximately 34 dB.

After applying gain limits a filtering of the table data is carried out. The filtered table data is displayed by trace 2. The filter is a multiple order linear phase FIR average filtration. The filter step is used to avoid sharp gain changes that can cause audible artifacts such as clicking noises.

In the final fifth step of the method adjacent filter gain contributions are managed. As will be discussed later, filter bandwidths cannot be very narrow and consequently gain leakage between the filters will occur. The stringent time domain requirements put on the filters mandates filter bandwidths that are wide enough to produce significantly more than zero dB gain at adjacent frequencies. For example, consider filters at two adjacent frequencies 1 kHz and 2 kHz. At each of these frequencies, 6 dB of amplification is required to restore a measured hearing loss. The filter at 1 KHz is set to add a gain at 1 kHz of +6 dB and the other filter at 2 kHz is set to add a gain at 2 kHz of +6 dB.

Unfortunately, the filter at 1 KHz produces a gain of +2 dB at 2 kHz and the filter at 2 kHz has a gain of +2 dB at 1 KHz. Simply setting the gain to +6 dB at each frequency will end up producing +8 dB total summed gain at both 1 kHz and 2 kHz due to the gain leakage from one filter to the other adjacent frequency. If the filters are ordinary second order parametric equalizers with Q of 0.96 and center frequencies of 1 kHz and 2 kHz respectively, which provides +2 dB gain at the adjacent frequency as just described, a gain setting of roughly 4.5 dB would produce the desired +6 dB gain at 1 KHz and 2 KHz.

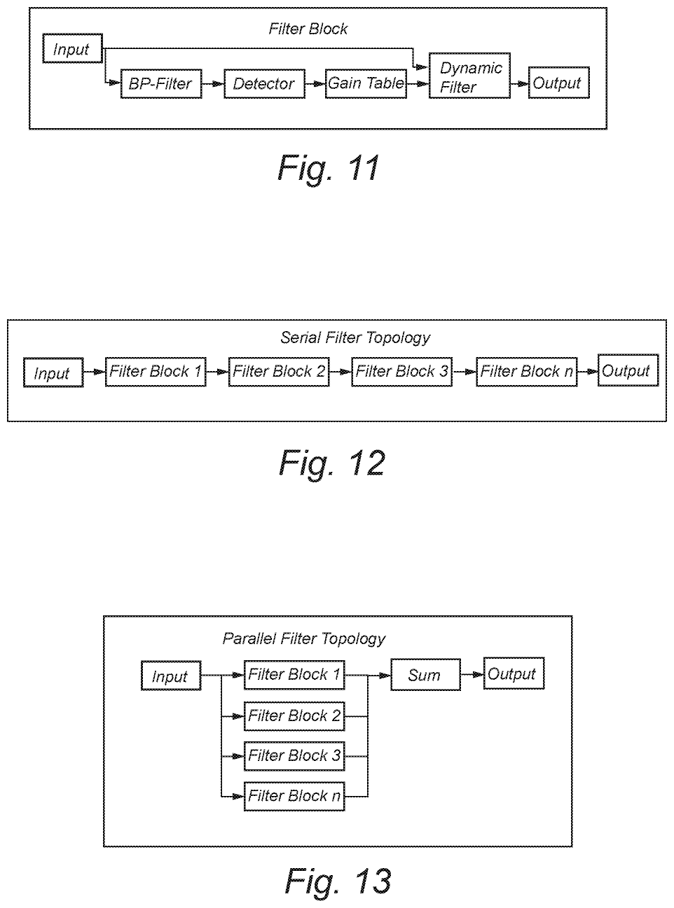

FIG. 19 shows a 3D graph of frequencies on the left axis, sound pressure level on the right axis and required amplification on the height axis. The height axis displays required amplification needed to restore a measured hearing loss at each frequency and sound pressure level. The dynamic gain behavior in relation to sound pressure level and frequency of the hearing restoration system can be studied in the diagram. There are seven individual boost frequencies in the diagram. Each of the boosts is provided by a Filter Block, FIG. 11, and a Dynamic Filter within the Filter Block. The filters producing gain at each of the FIG. 19 diagram frequencies will as discussed have to be wideband filters that stretches over into adjacent boost frequency bands. Therefore, adjacent frequency filter boost must be accounted for, or the aggregate gain will become significantly too large.

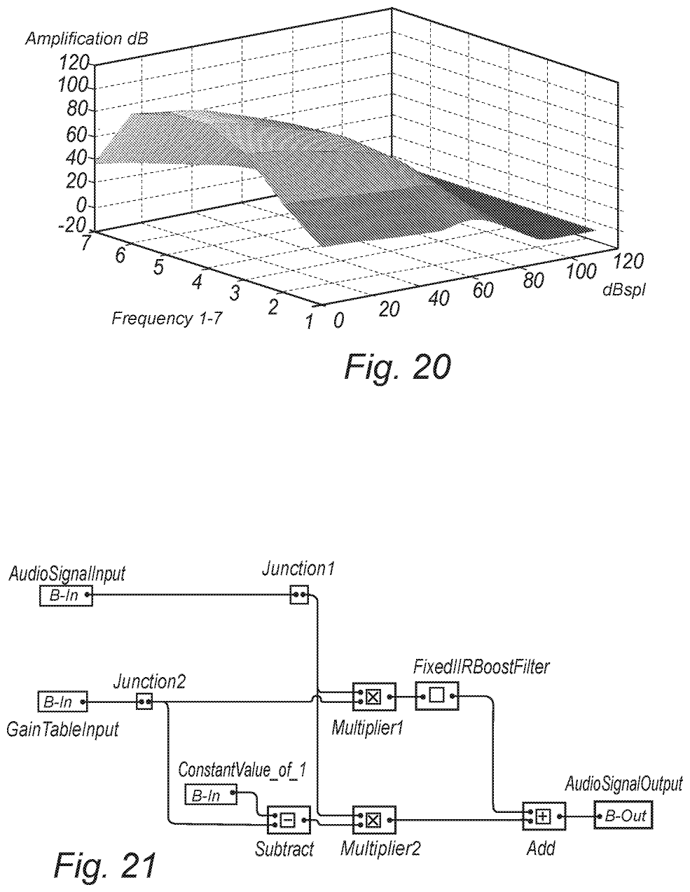

FIG. 20 shows gain summation from adjacent filters with realistic and suitable bandwidths without taking the aggregate gain into account. Comparing levels in FIG. 20 with the desired levels in FIG. 19 it becomes abundantly clear that the aggregate amplification produces far too much gain, upward of +90 dB in some areas where it only should have been around +35 dB. Clearly, taking adjacent filter gain contributions into account when producing the gain tables is very important.

Table amplification data and filter center frequencies are imported from the software algorithms to the DSP part. The DSP part contains one or more filter blocks providing dynamically varying amplification, each handling an individual frequency range. FIG. 11 shows an example filter topology that contains from the left a Band Pass Filter that limits the frequency range before the Level Detector, a Level Detector that detects the momentary input sound pressure level, a Gain Table that uses table data from the algorithms and translate the current input level to a gain setting in the Dynamic Filter. The Dynamic Filter amplifies sound within the Filter Block's frequency band according to Gain Table data input.

The filter block can of course be built using topologies other than what is shown in FIG. 11 and the example is just one of many possible implementations.

Other means than the mentioned Gain Tables can obviously be used to determine the Dynamic Filter's sound pressure level dependent gain. It is easy to understand that a polynomial or some other type of mathematical expression can be used as a substitute and the Gain Tables merely serve as an example of a feasible implementation approach. The Gain Table shown in FIG. 11 would in such case be substituted by the applied alternative method used to obtain the Dynamic Filter gain.

A Dynamic Filter is required at each frequency that needs to be amplified within the hearing restoration process. The dynamic filter is operating with dynamically changing gain dependent on the dynamically changing input signal level at the filter frequency. Many basic filter topologies are conceivable for the Dynamic Filter. For example, IIR topologies, FIR topologies etc. or combinations thereof. Regardless of the type of filter there is always a direct relationship between filter bandwidth and time domain response. A filter with narrower bandwidth will have poorer time domain response compared to a filter with wider bandwidth. As the bandwidth of the filter is reduced, progressively there will be more ringing present on the filter output. Also, the output response after a transient input signal to the filter will be more sluggish and delayed by a narrower bandwidth filter than a wider band filter. The filter order will also have a similar influence on the time domain response, where higher order filters produce better stopband attenuation or passband gain but exhibit poorer time domain behavior.

Human hearing is sensitive to time domain anomalies and filters with significant ringing and slow response times cause clearly audible sound degradation. Sound degradation is obviously not acceptable in a product and system aiming to improve hearing and sufficiently wide band filters are therefore required. If the 1 kHz and 2 kHz filters discussed before were narrow enough so that each of them only contributed 0.1 dB of gain at the adjacent frequency, they would have to have a Q of about 5.3 at 6 dB gain. This is a high Q causing significant ringing beyond 10 ms on the filter output after an input signal step and it will audibly clearly degrade sound. Filters with much lower Q values are therefore needed and filters with Q below 1 are behaving much better, the only significant drawback being that adjacent filter gain contributions must be compensated for.

Similarly to the Dynamic Filter, the time domain behavior of the Band Pass filter used before the Level Detector is important. The Band Pass filter is required to measure the signal level at the Dynamic Filter frequency and suppress sound signals present at other frequencies. A high Q filter output would not track the input signal well and detected levels would not be accurate due to filter output ringing and slow response. The Band Pass filters therefore must be of low order, preferably second, and with Q below 1. Functionally, there is also a benefit if the Band Pass filter and the Dynamic filter has matching bandwidths.

Building Dynamic filters in the digital domain using standard building blocks whilst preserving signal fidelity is not straight forward. The ubiquitously used infinite impulse response, IIR, and finite impulse response, FIR, filters do not behave predictably and well with dynamically changing filter coefficients. Nor does any combination of these filters. An IIR filter, regardless of chosen DSP implementation topology, operates through nested feedback paths with different delay lengths and coefficients used throughout for multiplications. For example, the common IIR bi-quadratic building block comprises five coefficients and four delay elements holding data from previous sample cycles. Any input signal other than zero to an IIR filter produce an impulse response on the output that tails off over time. The tail time is by definition infinite but in practice the output response will eventually fall into the noise floor, either through round of errors in the DSP-calculation's bit depth limited precision or an actual noise floor present in the input signal. The length of the tailing signal depends on the chosen filter order and Q, higher order and higher Q filters having longer tails. A FIR filter has within its filter structure similar delay elements holding data from previous samples and a multitude of coefficients. The filter does not rely on nested feedback loops, it uses a finite number of delay elements and coefficients defined by the filter length. The finite length, number of samples, of the filter also limits the tailing response to the same finite length of samples. Although a finite tailing response could sound beneficial a significant drawback with FIR filters compared to IIR is the latency they introduce. A FIR filter always delays the output signal by its number of samples finite length, which could be very troublesome in real time applications such as a hearing restoration system if the filter is long. Humans are sensitive to latency, even a few tenths of milliseconds is clearly perceptible and sounds that doesn't occur at the correct time in relation to visual impressions produces a quite strange and confusing feeling which is unacceptable for the application.

The tail on the output of any filter after the input signal has become zero is a measure of the energy stored in the delay elements within the filter. The filter models and formulas used to calculate filter coefficients from parameters assumes that the filter structure initially is in a steady state of zero, which, after an input signal has gone to zero, it only reaches subsequent to processing possibly hundreds of samples due to the nested feedback paths with different delay lengths or the filter length in case of a FIR filter. Regardless of whether it is an IIR, or FIR filter the stored energy within the filter is similar and the number of samples required to reach steady state of zero within the filter is more or less the same.

In a real time hearing restoration application, filters will never reach steady state zero within themselves since there will always be an input signal present. Filter parameter updates will always be done whilst energy is present within the filter structure. If filter parameters were updated occasionally, say once every ten seconds, this would not be a major issue, but a dynamic filter must be updated much more often, potentially every sample. Updating coefficients at a rate not far removed from or even approaching the sample rate will produce significant distortion. Updated coefficients will be multiplied with old data stored in the filter and consequently the output from the filter will become erroneous for as long as it takes for the stored data to propagate out through the filter structure. If coefficients are updated every sample the distortion will become very substantial.

Filters built on the Digital Integrator Cascades technique developed by Hal Chamberlin are an alternative to the IIR and FIR filters in dynamic applications. These filters are better suited to dynamic updates with much less distortion, but the filter responses are less accurate when resonances are introduced. The basic integrator form of these filters can only produce a limited set of filters and second order filters with Q values i.e., resonances, are not as accurate. Therefore, although commonly used in real time dynamic applications such as gaming software, their properties are less ideal in a hearing restoration system.

FIG. 21 shows a feasible implementation of the Dynamic Filter block present in FIG. 11 using an alternative structure that does not possess the drawbacks caused by dynamic updates of filter coefficients discussed above. The structure uses a fixed IIR filter, FixedIIRBoostFilter, that adds gain at the desired boost frequency. This filter can obviously be substituted with another type of filter or a combination of filters, the IIR filter is only a suitable example. The filter's coefficients are statically set so that maximum required boost is always provided by the filter. As an example, the gain table in FIG. 18 shows that a maximum gain of approximately 34 dB is required and consequently the filter gain would be set to 34 dB in this case. To the left in FIG. 21 are the input signals to the Dynamic Filter structure, AudioSignalInput and GainTableInput. The AudioSignalInput is obviously the audio input signal and the GainTableInput is the gain control input to the Dynamic Filter. The gain control input signal varies between zero and one depending on the required dynamically changing gain. When the signal is one, the Dynamic Filter produces maximum gain on the output and when zero it just passes through the audio signal without any gain at all being applied. The gain control signal is fed to Multiplier1 that is multiplying the audio input signal with the gain control signal. The gain control signal is also after being subtracted from the constant value of one fed to Multiplier2. The gain control signal after the subtraction varies between one and zero i.e., when the gain control signal to Multiplier1 is one the signal to Multiplier2 is zero and when zero to Multiplier1 it is one into Multiplier2. The audio signal output from Multiplier1 is fed to the FixedIIRBoostFilter which applies maximum gain and the output from the FixedlIRBoostFilter is fed to an adder, Add, summing the signal with the output form Multiplier2. The sum of the audio signal outputs from Multiplier1 and Multiplier2 will always add up to the same as the input audio signal, the ratio however between what goes out of Multiplier1 and Multiplier2 changes depending on the gain control input signal to the Dynamic Filter. By changing the gain control input signal to the Dynamic Filter a different mix of the unmodified but gain controlled output from Multiplier2 is added together with the boosted output from Multiplier1. By changing the signal ratio through the gain control input signal to the Dynamic Filter the boost provided by the Dynamic Filter can be varied from zero to maximum boost. This is achieved through changes of a simple mix of two audio signals entirely without any added distortion.

In many cases the DSP part will use more than one Filter Block and there are two obvious ways to combine Filter Blocks, a serial topology or a parallel. FIG. 12 shows an example of a serial topology and FIG. 13 reveals a parallel topology. Many combined Filter Block compositions can of course be constructed using other topologies than those shown in FIGS. 12 and 13.

Although there are two frequencies, 200 Hz and 800 Hz, in the Hearing Correction System example described that have amplification increasing with level most cases will have decreasing amplification when level increases. This dynamic reduction of amplification at higher input levels provides a clear benefit in hearing aid and similar applications where there always is a risk of acoustic feedback. If there is feedback and the level at the feedback frequency increases the amplification will automatically be reduced consequently reducing feedback and limiting it to a low level. In comparison, a fixed gain system will just increase the feedback until it reaches maximum output from the system which obviously can be very unpleasant and need to be aggressively prevented and avoided.

All modern hearing aids use feedback reduction techniques of various kinds. An aggressive feedback reduction system risk producing audible irregularities reducing the sound quality of the hearing aid. The more aggressive the greater the risk. The novel Hearing Correction System's automatic reduction of gain at higher levels put less burden on the feedback reduction system and it does not need to be as aggressive as would be required in a system with constant gain.

The described Hearing Correction System is not just applicable on hearing aids but can be used together with any sound reproduction system. Such a system can use any type of loudspeakers or headphones. The Hearing Correction System will function properly if the output level at the listener's ear is known i.e., a certain sound reproduction system output produces a known sound pressure level experienced by the listener. Most applications for the Hearing Correction System will for real time applications utilize some form of real time digital signal processing. It is however perfectly possible to preprocess audio material with the Hearing Correction System's compensation thereby creating a library of personally compensated audio.

Digital signal processing can be implemented in many ways, from pure hardware implementations to pure software/firmware or a mix between the two. The DSP functions in the described example use code written for a digital signal processor. The described hearing test and algorithms that produce input data to the DSP part is implemented in software running on a personal computer. The software could of course be implemented to run on any computing system such as a phone, tablet, or other device. It can also be implemented on a purpose built target system resembling an audiometer for the new Hearing Correction System.

The Digital signal processing part of the system will typically in real time applications be physically implemented within a hearing aid or headphone. I could also be implemented in a phone, tablet, TV-set, headphone amplifier, or computer to enhance call quality and sound reproduction in general using any regular headphones. It can also be implemented within a sound reproduction system in a car where the relationship between system output and sound pressure at the listener's ears is predictable due to the generally fixed physical location of a person within the car cabin.

All these arrangements are only provided as examples and many other possible implementation scenarios can obviously be envisaged.

### Embodiments of the Present Invention

Below some embodiments of the present invention are provided and discussed further.

According to one embodiment, said one or more dynamic filters are dynamically changed based on an in-signal and at least one parameter, preferably said one or more dynamic filters change the amplification depending on a change in sound pressure.

According to one embodiment, input sound pressure is amplified so that the experienced sound level is fully compensated.

Moreover, according to yet another embodiment, input sound pressure is amplified so that the experienced sound level is fully compensated within the limits set by acoustic feedback.

Furthermore, according to one embodiment, the method also comprises the step of processing and compensating the data to provide more or less equal loudness at each used frequency at different sound levels.

According to yet another embodiment, multiple dynamic filters are involved for compensating the data, more preferably a number of from 2-20 dynamic filters are involved for compensating the data.

As should be understood from above, suitably the method includes digital signal processing. Furthermore, according to yet another embodiment, the method involves determining dynamically changing amplification for each filter frequency. This step may involve either increasing and/or decreasing the amplification, depending on the measured data and input sound pressure level.

According to yet another embodiment of the present invention, the method involves determining dynamically changing amplification for each filter frequency.

Furthermore, according to one embodiment, table data is collected by drawing a straight vertical line at a particular center frequency creating table data from deviations between experienced level and real level, preferably with amplification of input signal at a certain frequency at different input sound pressures.

Moreover, according to one embodiment, the method includes digital signal processing of amplification data and filter center frequencies, preferably by involving one or more filter blocks.

The test according to the present invention may be used to identify frequencies/frequency range which need to be adjusted, and the filters are then used to adjust these. The center frequencies may be fixed or may be adjusted based on the measured data.

According to yet another embodiment of the present invention, the digital signal processing includes one or more filter blocks, preferably each filter block handling an individual frequency.

Moreover, according to yet another embodiment, each dynamic filter involved has a center frequency in a range of 200 Hz-12.8 KHz.

Furthermore, according to one embodiment, said one or more dynamic filters are wideband filters with low order, preferably second order, and wherein the method involves compensation for adjacent frequency filter boost to ensure a control of obtained aggregate gain.

Moreover, according to yet another embodiment, there is provided a dynamic filter for each frequency that needs to be amplified within the hearing restoration process. Furthermore, according to one embodiment, said one or more dynamic filters is operating with dynamically changing gain dependent on a dynamically changing input signal level at a certain filter frequency.

According to another embodiment of the present invention, maximum amplification is determined for one or more frequencies, preferably for each dynamic filter's center frequency, more preferably aggregate amplification from adjacent filter bands is also considered.

Each filter has a certain bandwidth and different filter's bandwidth may overlap. For example, if filter 1 has a center frequency of 1 kHz and filter 2 has 2 kHz, then filter 1 may contribute with certain amplification of frequencies at 2 kHz even if this is a considerably lower amplification than at 1 KHz. It is in this regard aggregate amplification may be needed according to the present invention.

Moreover, according to yet another embodiment of the present invention, maximum amplification is limited to avoid acoustic feedback.

The filter blocks may comprise different components according to the present invention. According to one embodiment, said one or more filter blocks comprise a band pass filter, a sound pressure detector, and a dynamic filter. According to one embodiment, the band pass filter is arranged to filter out and the detector is arranged to measure the signal level at the dynamic filter frequency and suppress sound signals present at other frequencies.

Furthermore, according to yet another embodiment, the band pass filter has a low order, preferably second order, more preferably with Q below 1.

Moreover, said one or more filter blocks may comprise a gain table. A gain table may be implemented as one possible way for transferring from sound pressure to gain. It should, however, be noted that other alternatives are possible according to the present invention. Moreover, according to yet another embodiment, multiple filter blocks are used. These may be arranged in series or parallel, or in combinations thereof.

Moreover, according to yet another embodiment, the method comprises transposing phon-related data into spl-related data.

Furthermore, and as exemplified above in relation to FIGS. 14-18, the method according to the present invention may also involve a numerical method for smart data handling outside of the measured area. In line with this, according to one embodiment, the method involves extending a model beyond obtained measured test data points by providing a fitted curve between at least multiple measured test data points, then providing an interpolation of the obtained fitted curve, and then providing a derivate fitted curve of the interpolation, preferably providing a first derivative fitted curve at a comparatively lower sound pressure level of the interpolation and a second derivative fitted curve at a comparatively higher sound pressure level of the interpolation. Moreover, according to yet another embodiment, the method involves a filtration step for removal of data points of the obtained measured test data points that are outside of a relationship for the obtained measured test data points, for enabling provision of a fitted curve between at least multiple measured test data points.

According to yet another embodiment, the present invention refers to a hearing correction system comprising:
a hearing test arranged to perform a hearing test with different frequencies and sound pressure levels on a test subject, said hearing test involving using a reference sound and a test sound and adjusting the test sound level until the test subject perceives the test sound level to be more or less equal to the reference sound in loudness, to obtain data on the test sound level and the reference sound for the different frequencies and sound levels;
a data processing unit arranged to process the obtained data and provide settings to a digital signal processing unit; and
the digital signal processing unit arranged to perform dynamic compensation,
wherein one or more dynamic filters are involved for the dynamic compensation of the data.

It should be noted that the hearing correction system according to the present invention may be implemented as a software unit in any type of suitable hardware device, such as a mobile phone, PC or other computer unit, tablet etc. Moreover, personal sound units, such as personal sound amplifier products are also such embodied by the present invention.

Furthermore, the digital signal processing unit may be based on software, hardware or a combination thereof.

Furthermore, according to yet another embodiment of the present invention, there is provided a system comprising a hearing correction system according to the present invention and a hearing aid unit, headphones or a sound reproduction system. An example of a sound reproduction system is loudspeakers.

Furthermore, the present invention is also directed to a method for hearing assessment and/or correction, said method comprising
performing a hearing test with different frequencies and with different sound levels on a test subject, said hearing test involving using a reference sound and a test sound and adjusting the test sound level until the test subject perceives the test sound level to be more or less equal to the reference sound in loudness; and
obtaining data on the test sound level and the reference sound for the different frequencies at different sound levels,
and wherein one or more dynamic filters are involved for compensating the data.

Furthermore, all embodies presented above in relation to a device according to the present invention, should also be seen as possible embodiments in relation to a method for hearing assessment and/or correction according to the present invention.

1. A device for hearing assessment and/or correction, said device comprising a software unit arranged for performing a method comprising:
performing a hearing test with different frequencies and with different sound levels on a test subject, said hearing test involving using a reference sound and a test sound and adjusting the test sound level until the test subject perceives the test sound level to be more or less equal to the reference sound in loudness; and
obtaining data on the test sound level and the reference sound for the different frequencies at different sound levels,
and wherein one or more dynamic filters are involved for compensating the data.

2. The device according to claim 1, wherein said one or more dynamic filters are dynamically changed based on an in-signal and at least one parameter, preferably said one or more dynamic filters change the amplification depending on a change in sound pressure.

3. The device according to claim 1, wherein input sound pressure is amplified so that the experienced sound level is fully compensated.

4. The device according to claim 3, wherein input sound pressure is amplified so that the experienced sound level is fully compensated within the limits set by acoustic feedback.

5. The device according to claim 1, wherein the method also comprises the step of:
processing and compensating the data to provide more or less equal loudness at each used frequency at different sound levels.

6. The device according to claim 1, wherein multiple dynamic filters are involved for compensating the data, preferably a number of from 2-20 dynamic filters are involved for compensating the data.

7. The device according to claim 1, wherein the method includes digital signal processing.

8. The device according to claim 6, wherein the method involves determining dynamically changing amplification for each filter frequency.

9. The device according to claim 6, wherein table data is collected by drawing a straight vertical line at a particular center frequency creating table data from deviations between experienced level and real level, preferably with amplification of input signal at a certain frequency at different input sound pressures.

10. The device according to claim 1, wherein the method includes digital signal processing of amplification data and filter center frequencies, preferably by involving one or more filter blocks.

11. The device according to claim 7, wherein the digital signal processing includes one or more filter blocks, preferably each filter block handling an individual frequency.

12. The device according to claim 1, wherein each dynamic filter involved has a center frequency in a range of 200 Hz-12.8 KHz.

13. The device according to claim 6, wherein maximum amplification is determined for one or more frequencies, preferably for each dynamic filter's center frequency, more preferably aggregate amplification from adjacent filter bands is also considered.

14. The device according to claim 1, wherein maximum amplification is limited to avoid acoustic feedback.

15. The device according to claim 1, wherein said one or more dynamic filters are wideband filters with low order, preferably second order, and wherein the method involves compensation for adjacent frequency filter boost to ensure a control of obtained aggregate gain.

16. The device according to claim 1, wherein there is provided a dynamic filter for each frequency that needs to be amplified within the hearing restoration process.

17. The device according to claim 1, wherein said one or more dynamic filters is operating with dynamically changing gain dependent on a dynamically changing input signal level at a certain filter frequency.

18. The device according to claim 10, wherein said one or more filter blocks comprise a band pass filter, a sound pressure detector, and a dynamic filter.

19. The device according to claim 18, wherein the band pass filter is arranged to filter out and the detector is arranged to measure the signal level at the dynamic filter frequency and suppress sound signals present at other frequencies.

20. The device according to claim 18, wherein the band pass filter has a low order, preferably second order, more preferably with Q below 1.

21. The device according to claim 20, wherein said one or more filter blocks also comprise a gain table.

22. The device according to claim 1, wherein the method comprises transposing phon-related data into spl-related data.

23. The device according to claim 10, wherein multiple filter blocks are used.

24. The device according to claim 1, wherein the method involves extending a model beyond obtained measured test data points by providing a fitted curve between at least multiple measured test data points, then providing an interpolation of the obtained fitted curve, and then providing a derivate fitted curve of the interpolation, preferably providing a first derivative fitted curve at a comparatively lower sound pressure level of the interpolation and a second derivative fitted curve at a comparatively higher sound pressure level of the interpolation.

25. The device according to claim 24, wherein the method involves a filtration step for removal of data points of the obtained measured test data points that are outside of a relationship for the obtained measured test data points, for enabling provision of a fitted curve between at least multiple measured test data points.

26. A hearing correction system comprising:
a hearing test arranged to perform a hearing test with different frequencies and sound pressure levels on a test subject, said hearing test involving using a reference sound and a test sound and adjusting the test sound level until the test subject perceives the test sound level to be more or less equal to the reference sound in loudness, to obtain data on the test sound level and the reference sound for the different frequencies and sound levels;
a data processing unit arranged to process the obtained data and provide settings to a digital signal processing unit; and
the digital signal processing unit arranged to perform dynamic compensation, wherein one or more dynamic filters are involved for the dynamic compensation of the data.

27. The system according to claim 26, wherein the digital signal processing unit comprises software, hardware or a combination thereof.

28. A system comprising a hearing correction system according to claim 26 and a hearing aid unit, headphones or a sound reproduction system.

29. A method for hearing assessment and/or correction, said method comprising:
performing a hearing test with different frequencies and with different sound levels on a test subject, said hearing test involving using a reference sound and a test sound and adjusting the test sound level until the test subject perceives the test sound level to be more or less equal to the reference sound in loudness; and
obtaining data on the test sound level and the reference sound for the different frequencies at different sound levels,
and wherein one or more dynamic filters are involved for compensating the data.
